(19) United States
(12) Patent Application Publication
(10) Pub. No.: US 2011/0052699 A1
(43) Pub. Date: Mar. 3, 2011

(54) DRUG DELIVERY SYSTEM WITH STABILISING EFFECT

(76) Inventors: Adrian Funke, Berlin (DE); Ildiko Terebesi, Berlin (DE); Sascha General, Berlin (DE)

(21) Appl. No.: 12/867,165

(22) PCT Filed: Feb. 10, 2009
(86) PCT No.: PCT/EP2009/000904

§ 371 Date: Nov. 17, 2010

(30) Foreign Application Priority Data
Feb. 13, 2008 (EP) 08002633.9
Aug. 4, 2008 (EP) 08162105.4
Nov. 21, 2008 (EP) 08105842.2

(51) Int. Cl. A61K 9/14; A61K 31/585; A61K 31/565; A61K 31/57; A61P 5/30

(52) U.S. Cl. 424/486; 514/170; 424/485; 514/182; 514/178

(57) ABSTRACT

A drug delivery system also intended as unit dosage form comprising a thin water-soluble film matrix, wherein said film matrix comprises
a) a polyvinyl alcohol-polyethylene glycol graft copolymer (PVA-PEG graft co-polymer) as a water-soluble matrix polymer;
an active ingredient being a steroid in which the positions 6 and 7 of the steroidal skeleton are both a —CH₂— residue; and
   said film matrix has a thickness of less than 300 μm.

### FIELD OF THE INVENTION

The present invention relates to drug delivery systems in the form of thin water-soluble films (wafers), which contain as an active ingredient at least a steroid in which the positions 6 and 7 of the steroidal skeleton are both a —CH₂— residue, in particular said at least one steroid being an estrogen. The present invention further refers to a drug delivery system comprising an estrogen, a progestin or a combination thereof as active ingredients and in which at least one of said active ingredients is a steroid in which the positions 6 and 7 of the steroidal skeleton are both a —CH₂— residue.

The drug delivery systems of the present invention further refer to wafers comprising an estrogen as active ingredient, like for example estradiol, ethinylestradiol or an Estrogen Receptor β (ERβ) selective agonist, particularly a 8β- or 9a-substituted estra-1,3,5(10)-triene as ERβ selective agonist in which said estrogen is a steroid in which the positions 6 and 7 of the steroidal skeleton are both a —CH₂— residue.

The drug delivery systems according to the invention further refers to wafers comprising an estrogen or a progestin or a combination thereof which can be favourable used as a medicament.

The delivery systems according the invention, also intended as a unit dosage forms, comprise a polyvinyl alcohol-polyethylene glycol graft copolymer (PVA-PEG graft co-polymer, or simply PVA-PEG copolymer) as a water-soluble matrix polymer. The delivery systems, to, which the present invention refers, provide an improved stability to one or more of the active ingredients comprised therein. The present invention further refers to a drug delivery system in the form of thin water-soluble film (wafer) with an improved mouthfeel. The wafers of the present invention are particularly used for the formulation of steroidal hormones, particularly steroidal sex hormones, like estrogens, progestins or a combination thereof.

### BACKGROUND OF THE INVENTION

Several active ingredients are known to have limited stability to oxidation processes at ambient conditions (such as 25° C./60% relative humidity) or accelerated storage conditions (such as 40° C./75% relative humidity) and undergo transformations which can cause an alteration of the effective amount of active ingredient and sensitively influence their bioequivalence profile.

Hormones are usually formulated in very little amounts, for this reason also very little variations in amount of the active ingredient within the pharmaceutical formulation can dramatically influence the desired effect for said pharmaceutical products. The oxidative degradation of estrogens and progestins is well known in the field and is typical an issue with reference to the shelf life of the related solid preparation (T. Hurley et al, “Norethindrone acetate (NA) and ethinyl estradiol (EE) related oxidative transformation products in stability samples of formulated drug product: synthesis of authentic references”, Steroids, Vol. 67 (2002), pages 165-174; Van D. Reif et al., “Automated Stability-Indicating High-Performance Liquid Chromatographic Assay for Ethinyl Estradiol and (Levo)norgestrel Tablets”, Pharmaceutical Research, Vol. 4 (1987), pages 54-58).

In WO96/02277A1a method and pharmaceutical compositions are disclosed for reducing oxidative degradation of 17 alpha-ethinylestradiol comprising combining the estradiol with an effective amount of cyclodextrin, thus forming a cyclodextrin clathrate of the steroid. The above patent document particular relates to solid dosage forms that contain steroidal sex hormones. It is reported that natural and especially synthetically derived sex hormones are generally highly effective active ingredients of pharmaceutical agents. Consequently, in most cases solid dosage forms contain these active ingredients at very low dosages; these are usually well lower than 1 mg per single-dosed dosage form. This means that both the preparation and the stability during storage and use of these dosage forms are often problematical in nature.

As discussed above, in the preparation of such low-dosed dosage forms, strong fluctuations of the active ingredient concentrations in the dosage units occur almost unavoidably (inadequate content uniformity), which manifest themselves more strongly, the smaller the amount of the active ingredient.

In the storage of such low-dosed preparations, moreover, a reduction in the active ingredient concentration is often additionally observed as a result of, in most cases, oxidative degradation reactions of the active ingredient.

In addition, at such low dosage the bioavailability of the active ingredient is subject to a pronounced first-pass effect and exhibits great inter- and intra-individual fluctuations.

However, while drugs, such as estrogens, may be included in traditional standard oral tablet or capsule formulations to provide an accurate and consistent dose, such delivery forms have several disadvantages in both the administration and preparation of the drug. For example, it has been estimated that about 50% of the population have problems swallowing tablets (see Seager in J. Pharmacol. Pharm 1998; 50; 375-382), and patients such as children or the elderly who will not, or cannot, swallow tablets or capsules represent a challenge for the pharmaceutical industry. The pharmaceutical industry has tried to meet this challenge by developing a number of different drug delivery systems, including rapid in-mouth disintegrating tablets, tablets which disintegrate in liquid prior to ingestion, liquids and syrups, gums and even transdermal patches. However, each of these drug delivery systems can pose their own problems.

Rapid in-mouth disintegrating tablets, such as chewable or self disintegrating tablets offer great convenience. However, chewable or self-disintegrating tablets often present real taste masking problems as the act of chewing can disrupt protective coatings. Furthermore, chewable or self-disintegrating tablets are often associated with an unpleasant mouthfeel. Moreover, the fear of swallowing, chewing, or choking on such solid shaped articles is still a concern in certain populations. In addition, the fragility/friability of such porous, and low-pressure molded tablets makes them difficult to carry, store, handle and administer to patients, especially the children and the elderly.

Chewable taste-masked pharmaceutical compositions are described for example in U.S. Pat. No. 4,800,087. Taste-masked orally disintegrating tablets (ODTs) are described in US 2006/0105038. Taste-masking coating systems are described in WO00/30617. Taste-masked wafers are described in WO 03/030883.

Disintegrating buccal tablets which contain a physiologically active material and a vinyl alcohol/polyethylene glycol graft copolymer are described in the patent document WO2006/029787A1; together with a method for the production of disintegrating buccal tablets which is characterized in that after granulating a composition which contains a physiologically active material and a vinyl alcohol/polyethylene glycol graft copolymer, tableting is performed.

The document WO2005/039499A2 describes disintegratable films containing a mixture of high molecular weight and low molecular weight water soluble components; and a pharmaceutically or cosmetically active ingredient. Optionally, the films contain a starch component, a glucose component, a filler, a plasticizer and/or humectant. The films are preferably in the form of a mucoadhesive monolayer having a thickness sufficient to rapidly disintegrate in the oral environment and release the active ingredient without undue discomfort to the oral mucosa. The monolayer can be cut to any desired size or shape to provide conveniently useable unit dosage forms for administration to oral or other mucosal surfaces for human pharmaceutical, cosmetic, or veterinary applications. WO2005/039499 further describes methods of administering the film compositions by placing the composition into, for example, the oral cavity for a sufficient period of time to permit the film to disintegrate and release the active ingredient. No explicit example with a polyvinyl alcohol-polyethylene glycol copolymer is specifically disclosed.

WO2005/009386A2 describes a rapidly dissolving, oral film preparations for rapid release of an active agent in the oral cavity, in particular, rapidly dissolving oral films comprising a nicotine active which achieve good transbuccal absorption and provide nicotine craving relief to an individual are disclosed herein. Examples of a dissolving film comprising polyvinyl alcohol-polyethylene glycol graft copolymer are described. However no reference is made to stability of such formulation over the active ingredient.

Finally, WO2007/115381A2 describes the use of a polyvinyl alcohol-polyethylene glycol graft copolymer (PVA-PEG graft co-polymer), such as Kollicoat IR, in the formulation of solid dispersions of low aqueous solubility and dissolution rate bioactive compound and, more particularly to a system and method for improving the solubility and dissolution rate of such low aqueous solubility and dissolution rate bioactive compound, in particular the drug of low aqueous solubility, such as a BCS Class II or Class IV drug compounds.

With reference to active ingredients which can be part of a drug delivery system according to the present invention, and particular in the case that said ingredient are selective steroidal estrogens, the attention is pointed to the fact that selective estrogens represent a newer alternative to estrogen/progestin combination products. Selective estrogens have to date been understood to be compounds having estrogen-like effects on brain, bone and vascular system because of their anti-uterotrophic (i.e. anti-estrogenic) partial effect, but not having a proliferative effect on the endometrium.

Estrogen receptor modulators with preference for ER-β, in particular ER-β selective agonists, may also have a beneficial effect on brain functions, bladder, intestine and the cardiovascular system without having in the same dose range a hepatic estrogen effect or stimulating effect on endometrium and breast, ER-β agonists therefore represent a novel option for selective estrogen therapy and for the treatment of hot flushes and mood fluctuations. The occurrence of hot flushes presumably derives from an instability of the hypothalamic thermoregulatory set point caused by the decline in estrogens and the onset of the menopause (Stearns V, Ullmer L, Loepez J F, Smith Y, Isaacs C, Hayes D F (2002) Hot flushes. The Lancet 360: 1851-1861).

WO 01/77139A1 describes 8β-substituted estratrienes wherein R⁸ means a straight-chain or branched-chain, optionally partially or completely halogenated alkyl or alkenyl radical with up to 5 carbon atoms, an ethinyl- or prop-1-inyl radical, as pharmaceutical active ingredients that have in vitro a higher affinity to estrogen receptor preparations of rat prostates than to estrogen receptor preparations of rat uteri, their production, their therapeutic use and pharmaceutical dispensing forms that contain the said compounds

WO 03/104253A2 relates to novel 9a-substituted estratrienes in which R⁹ represents a linear-chain or branched-chain, optionally partially or fully halogenated alkenyl radical comprising between 2 and 6 carbon atoms, or an ethinyl radical or a prop-1-inyl radical—as pharmaceutical active ingredients which have, in vitro, a higher affinity to estrogen receptor preparations of the rat prostate than to estrogen receptor preparation of the rat uterus, and, in vivo, preferably a preferential action on the ovary compared to the uterus.

PCT/EP2008/059115 refers 8β-substituted estra-1,3,5(10)-triene derivatives of general formula I, their use as pharmaceutical active ingredients, which have in vitro a higher affinity to estrogen receptor preparations from rat prostates than to estrogen receptor preparations from rat uteri and in vivo a preferential action in the ovary in comparison to the uterus, their production, their therapeutic use and pharmaceutical dispensing forms that contain the new compounds.

The above documents refer particularly to estrogens selective for ERβ. Routine test for identifying the selective ERβ activity in vitro and in vivo are reported in the documents cited above, for example on page 18 to 23 of WO03/104253A2.

A further routine test to identify compounds with selective ERβ activity, is as follows:

### Cellular In Vitro Assay to Determine the Estrogen Receptor-α and -β Activity

### Abbreviations:

DMEM Dulbecco's modified Eagle medium
DNA deoxynucleic acid
FCS fetal calf serum
HEPES 4-(2-hydroxyethyl)-1-piperazineethanesuiphonic acid
PCR polymerase chain reaction

Modulators of the human estrogen receptors-α and -β ERα and ERβ) are identified, and the activity of the substances described herein is quantified, with the aid of recombinant cell lines. These cells are originally derived from a hamster ovary epithelial cell (Chinese Hamster Ovary, CH0 K1, ATCC: American Type Culture Collection, VA 20108, USA).

An established chimera system in which the ligand-binding domains of human steroid hormone receptors are fused to the DNA-binding domain of the yeast transcription factor GAL4-are used in this CH0 K1 cell line. The GAL4-steroid hormone receptor chimeras produced in this way are cotransfected and stably expressed with a reporter construct in the CHO cells.

### Cloning:

To generate the GAL4-steroid hormone receptor chimeras, the GAL4 DNA-binding domain (amino acids 1-147) from the vector pFC2-dbd (from stratagene) is cloned with the PCR-amplified ligand-binding domains of the estrogen receptor α (ERα, Genbank accession number NM00125, amino acids 282-595) and of the estrogen receptor β (ERβ, Genbank accession number AB006590, amino acids 223-530) into the vector pIRES2 (from Clontech). The reporter construct, which comprises five copies of the GAL4 binding site upstream of a thymidine kinase promoter, leads to expression of firefly luciferase (Photinus pyralis) after activation and binding of the GAL4-estrogen receptor chimeras by specific agonists.

Assay procedure: the stock cultures of ERα and ERβ cells are routinely cultured in DMEM/F12 medium, 10% FCS, 1% Hepes, 1% penicillin/streptomycin, 1 mg/ml G418, and 5 μg/ml puromycin. On the day before the assay, the ERα and ERβ cells are plated out in Opti-MEM medium (Optimem, from Invitrogen, 2.5% activated carbon-purified FCS from Hyclone, 1% Hepes) in 96- (or 384) well microlitre plates and kept in a cell incubator (96% humidity, 5% v/v CO₂, 37° C.). On the day of the assay, the substances to be tested are taken up in the abovementioned medium and added to the cells. If it is intended to investigate possible antagonistic properties of test substances, the estrogen receptor agonist 17-β estradiol (from Sigma) is added 10 to 30 minutes after addition of the test substances, but no additional addition of 17-β estradiol takes place in the investigation of agonistic properties. After a further incubation time of 5 to 6 hours, the cells are lysed with a Iuciferin/Triton buffer, and the luciferase activity is measured with the aid of a video camera. The measured relative light units as a function of the substance concentration result in a sigmoidal stimulation curve. The EC50 and values are calculated with the aid of the GraphPad PRISM (version 3.02) computer program.

A similar test is also described in: Peekhaus Norbert T. et al., ASSAY and Drug Development Technologies Volume 1, Number 6, 2003 “A β-Lactamase-Dependent Gal4-Estrogen Receptor b Transactivation Assay for the Ultra-High Throughput Screening of Estrogen Receptor β Agonists in a 3,456-Well Format”

The patent documents WO01/77139A1, WO03/104253A2 and PCT/EP2008/059115 are incorporated by reference in the present application.

There is clearly a need in the present field of pharmaceutical formulations of drug delivery systems in the form of thin water-soluble films (wafers), able of conferring an improved stability to the active ingredient contained therein for example in the case the active ingredient is affected by oxidative degradation.

Furthermore pharmaceutical formulations directed to the oral cavity, and in particular wafer with improved mouthfeel are highly desirable in the pharmaceutical field particularly with reference to a higher acceptability in a long term use.

### SUMMARY OF THE INVENTION

In a first aspect, the present invention relates to a unit dosage form comprising a thin water-soluble film matrix, wherein said film matrix comprises
a) a polyvinyl alcohol-polyethylene glycol graft copolymer (PVA-PEG graft co-polymer) as a water-soluble matrix polymer;
b) an active ingredient being a steroid in which the positions 6 and 7 of the steroidal skeleton are both a —CH₂— residue; and
   said film matrix has a thickness of less than 300 μm.

According to a particular form of embodiment, the dosage form according to the invention comprises as an active ingredient a steroidal estrogen in which the positions 6 and 7 of the steroidal skeleton are both a —CH₂— residue, and more particularly in which said steroidal estrogen comprises an hydroxy, an ester or an ether group in position 3 of the steroidal skeleton.

In particular, the steroidal estrogen as defined above can be selected from the group comprising ethinylestradiol, estradiol, estrone, mestranol, estriol, estriol succinate, estrone sulfate, 17β-estradiol sulfate, 17α-estradiol sulfate, estradiol valerate, including therapeutically acceptable derivates.

Furthermore, in an another embodiment of the unit dosage form according to the invention, said steroidal estrogen is a 8β- or 9α-substituted estra-1,3,5(10)-triene being a ERβ selective agonist. Particular examples for said ERβ selective agonist which can be part of the wafer according to the invention are
9a-Vinyl-estra-1,3,5(10)-triene-3,16a-diol,
17β-Fluoro-9a-Vinyl-estra-1,3,5(10)-triene-3,16a-diol,
18a-Homo-9a-vinyl-estra-1,3,5(10)-triene-3,16a-diol,
16a-Fluoro-8β-vinyl-estra-1,3,5(10)-triene-3,17a-diol,
16a-Fluoro-8β-vinyl-estra-1,3,5(10)-triene-3,17β-diol,
16β-Fluoro-8β-vinyl-estra-1,3,5(10)-triene-3,17β-diol,
8β-Vinyl-estra-1,3,5(10)-triene-3,17β-diol,
   or derivatives thereof.

In a further aspect, the present invention relates to a unit dosage form comprising a thin water-soluble film matrix (wafer) wherein said active ingredient is a steroidal progestin in which the positions 6 and 7 of the steroidal skeleton are both a —CH₂— residue.

In particular, the steroidal progestin can be further selected from the group comprising levonorgestrel, norgestrel, norethindrone (norethisterone), dienogest, norethindrone (norethisterone) acetate, ethynodiol diacetate, norethynodrel, allylestrenol, lynestrenol, norgestrienone, ethisterone, promegestone, desogestrel, 3-keto-desogestrel, norgestimate, gestodene.

In an other aspect of the present invention, said film matrix comprises an estrogen and a progestin as active ingredients, and at least one of said active ingredients is a steroid in which the positions 6 and 7 of the steroidal skeleton are both a —CH₂— residue.

According to a particular form of embodiment, in the wafer according to the invention, a steroidal estrogen in which the positions 6 and 7 of the steroidal skeleton are both a —CH₂— residue, is combined with a progestin which is a 16,17-carbolactone derivative, for example drospirenone.

In another aspect, the present invention relates to a unit dosage form for use as a medicament.

In a further aspect, the present invention relates to a unit dosage form to be used in Hormone Replacement Therapy (HRT) and in particular for treating, alleviating or preventing a physical condition in a female mammal caused by insufficient endogenous levels of estrogen. Examples of such physical conditions include, but is not limited to, osteoporosis, headaches, nausea, depression, vasomotor symptoms, symptoms of urogenital atrophy, decrease in bone mineral density, and increased risk or incidence of bone fracture.

The drug delivery systems in the form of thin water-soluble films (wafers) according to the present invention can be favourably used also for contraception.

Other aspects of the present invention will be apparent from the below description and the appended claims.

### DETAILED DESCRIPTION OF THE INVENTION

Herein, the term “active ingredient”, “drug substance”, “active drug substance” or simply “drug”, is intended to mean any pharmaceutically active compound comprised in the dosage form according to the present invention.

The “drug delivery systems” according to the invention are also intended within the meaning of “unit dosage forms” and vice versa.

The drug delivery systems in the form of thin water-soluble films (wafers) according to the invention, comprise particularly at least one steroid as active compounds, in which the positions 6 and 7 of the steroidal skeleton are both a —CH₂— residue, this means that positions 6 and 7 are not substituted.

In this regards, the term “steroidal skeleton” refers to the 4-rings system:

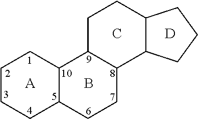

The wafers according to the invention particularly comprise steroids which are 8β- or 9a-substituted estra-1,3,5(10)-trienes. The backbone for said substituted steroids can be represented as follow:

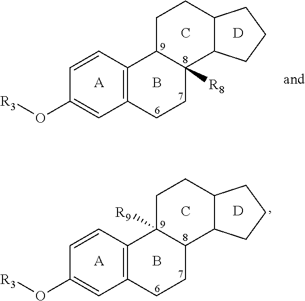

In the present context, the term “progestin” (also sometimes referred to as “gestagen” or “progestogen”) covers synthetic hormone compounds which are progesterone receptor agonists. The term is further meant to encompass all isomeric and physical forms of the progestins including hydrates, solvates, salts and complexes, such as complexes with cyclodextrins. Specific examples of progestins include, but are not limited to, progestins selected from the group consisting of 16,17-carbolactone derivatives (for example drospirenone), and levonorgestrel, norgestrel, norethindrone (norethisterone), dienogest, norethindrone (norethisterone) acetate, ethynodiol diacetate, dydrogesterone, medroxyprogesterone-acetate, norethynodrel, allylestrenol, lynestrenol, quingestanol acetate, medrogestone, norgestrienone, dimethisterone, ethisterone, chlormadinone acetate, megestrol, promegestone, desogestrel, 3-keto-desogestrel, norgestimate, gestodene, tibolone, and cyproterone acetate. Particularly preferred progestins are 16,17-carbolactone derivatives (for example drospirenone), and levonorgestrel, dienogest, gestodene, and cyproterone acetate.

Steroidal progestins in which the positions 6 and 7 of the steroidal skeleton are both a —CH₂— residue include, but are not limited to, compounds selected from the group consisting of levonorgestrel, norgestrel, norethindrone (norethisterone), dienogest, norethindrone (norethisterone) acetate, ethynodiol diacetate, norethynodrel, allylestrenol, lynestrenol, norgestrienone, ethisterone, promegestone, desogestrel, 3-keto-desogestrel, norgestimate, gestodene including therapeutically acceptable derivates.

As discussed infra, the progestin may be complexed with a cyclodextrin and/or combined with an protective agent.

The term “estrogen” is meant to encompass all natural or synthetic steroidal compounds exhibiting estrogenic activity. Such compounds encompass inter alia conjugated estrogens, and phytoestrogens. The term is further meant to encompass all isomeric and physical forms of the estrogens including hydrates, solvates, salts and complexes, such as complexes with cyclodextrins. More particularly, steroidal estrogens in which the positions 6 and 7 of the steroidal skeleton are both a —CH₂— residue include the estrogens selected from the group consisting of ethinylestradiol, estradiol including therapeutically acceptable derivates (including esters) of estradiol, estrone, mestranol, estriol, estriol succinate and conjugated estrogens, including conjugated equine estrogens such as estrone sulfate, 17β-estradiol sulfate, 17α-estradiol sulfate. Particular interesting estrogens are selected from the group consisting of ethinylestradiol, estradiol, estradiol sulfamates, estradiol valerate, estradiol benzoate, estrone, mestranol and estrone sulfate. More preferably, the estrogen is ethinylestradiol or estradiol. The most preferred estrogen is ethinylestradiol.

According to a particular embodiment of the present invention 8β- or 9a-substituted estra-1,3,5(10)-triene as ERβ selective agonist, more particularly a compound chosen from the group comprising:
9a-Vinyl-estra-1,3,5(10)-triene-3,16a-diol,
17β-Fluoro-9a-Vinyl-estra-1,3,5(10)-triene-3,16a-diol,
18a-Homo-9a-vinyl-estra-1,3,5(10)-triene-3,16a-diol,
16a-Fluoro-8β-vinyl-estra-1,3,5(10)-triene-3,17a-diol,
16a-Fluoro-8β-vinyl-estra-1,3,5(10)-triene-3,178-diol,
16β-Fluoro-8β-vinyl-estra-1,3,5(10)-triene-3,178-diol,
8β-Vinyl-estra-1,3,5(10)-triene-3,178-diol, or derivatives thereof are considered under the definition of estrogen.

Furthermore when used herein, the term “derivatives thereof” refers particularly to those esters of a 8β- or 9a-substituted estra-1,3,5(10)-triene which would be apparent to the pharmaceutical chemist, i.e. those which are substantially non-toxic and which may favourably affect the pharmacokinetic properties of the identified compounds, such as palatability, absorption, distribution, metabolism and excretion. Typically, an ester of the compounds related to the present invention is in the 3-position or 17-position of a 8β- or 9a-substituted estra-1,3,5(10)-triene defined above. Specific examples of pharmaceutically acceptable esters include valerate, acetate, propionate, enantate, undecylate, benzoate, cypionate, sulfate and sulfamate esters.

When used herein, the term “therapeutically acceptable derivative of estradiol” refers to esters of estradiol; salts, such as sodium salts, of estradiol and estradiol esters; as well as other derivatives known in the art. Typically, an ester of estradiol is in the 3-position or 17-position of estradiol. Specific examples of typical esters of estradiol include estradiol valerate, estradiol acetate, estradiol propionate, estradiol enantate, estradiol undecylate, estradiol benzoate, estradiol cypionate, estradiol sulfate, estradiol sulfamate, as well as salts thereof. Estradiol valerate is particularly preferred among the estradiol esters.

The term “estradiol” is intended to mean that the estradiol may be in the form of 17-α-estradiol or 17-β-estradiol. Preferably, the estradiol is in the form of 17-β-estradiol. The term “estradiol” also covers hydrated forms of estradiol, in particular estradiol hemihydrate.

As discussed infra, the estrogen may be complexed with a cyclodextrin and/or combined with an protective agent.

The term “water-soluble film matrix”, wherein used herein, refers to a thin film which comprises, or consists of, a water-soluble polymer and active ingredients as well as other auxiliary components dissolved or dispersed in the water-soluble polymer. In a preferred embodiment, at least one active ingredient is completely dissolved in the water-soluble polymer.

As used herein, the term “water-soluble polymer” refers to a polymer that is at least partially soluble in water, and preferably fully or predominantly soluble in water, or absorbs water. Polymers that absorb water are often referred to as being “water-swellable polymers”. The materials useful for the present invention may be water-soluble or water-swellable at room temperature (about 20° C.) and other temperatures, such as temperatures exceeding room temperature. Moreover, the materials may be water-soluble or water-swellable at pressures less than atmospheric pressure. Desirably, the water-soluble polymers are water-soluble, or water-swellable having at least 20% by weight water uptake. Water-swellable polymers having 25% by weight, or more, water uptake, are also useful. The unit dosage forms of the present invention formed from such water-soluble polymers are desirably sufficiently water-soluble to be dissolvable upon contact with bodily fluids, in particular saliva. In a preferred embodiment of the invention, the water-soluble polymer is a mucoadhesive polymer. This will allow for transmucosal delivery of the active ingredient, as a particular example a ERβ selective agonist, in case of a steroidal estrogen, and ensure efficient uptake of the molecule by avoiding the first pass metabolism. The water-soluble polymer typically constitutes from 50-99.99% by weight, such as from 75-99.9% by weight, of the water-soluble film matrix.

The water-soluble matrix polymer (constituting the major part of the water-soluble film matrix) according to the present invention comprises polyvinyl alcohol-polyethylene glycol graft co-polymers (PVA-PEG co-polymers), which are commercially available in different grades under the trademark Kollicoat® IR. Said PVA-PEG co-polymers constitute at least more than 50%, or 60%, or 70%, or 80%, or 90% by weight of the water-soluble film matrix according to the invention.

Preferably, the PVA-PEG co-polymers constitute more than 90% by weight, most preferably more than 95%.

The preferred PVA-PEG co-polymer is the one commercialised as Kollicoat® by the company BASF, Germany, which comprises 75% polyvinyl alcohol units and 25% polyethylene glycol units.

The water-soluble matrix polymer can further comprise other water-soluble matrix polymers such as those selected from the group consisting of a cellulosic material, a synthetic polymer, a gum, a protein, a starch, a glucan and mixtures thereof. Said other water-soluble matrix polymers typically constitute less than 40%, or 30%, or 20%, or 10% of the water-soluble film matrix. Preferably, said other water-soluble matrix polymers are less than 30% of the water-soluble film matrix

Examples of cellulosic materials suitable for the purposes described herein include carboxymethyl cellulose, methyl cellulose, ethyl cellulose, hydroxymethyl cellulose, hydroxyethyl cellulose, hydroxypropyl cellulose, hydroxymethylpropyl cellulose, hydroxypropylmethyl cellulose and combinations thereof. Particularly preferred cellulosic materials are hydroxypropylmethyl cellulose and hydroxy-propyl cellulose, in particular hydroxypropylmethyl cellulose.

Examples of others synthetic polymers which can be used in combination with the PVA-PEG co-polymers include polymers, such as polyacrylic acid and polyacrylic acid derivatives.

Examples of water-soluble gums include gum arable, xanthan gum, tragacanth, acacia, carageenan, guar gum, locust bean gum, pectin, alginates and combinations thereof.

Useful water-soluble protein polymers include gelatine, zein, gluten, soy protein, soy protein isolate, whey protein, whey protein isolate, casein, levin, collagen and combinations thereof.

Examples of useful starches include gelatinised, modified or unmodified starches. The source of the starches may vary and include pullulan, tapioca, rice, corn, potato, wheat and combinations thereof.

Additional water-soluble polymers, which may be used in accordance with the present invention, include dextrin, dextran and combinations thereof, as well as chitin, chitosin and combinations thereof, polydextrose and fructose oligomers.

As indicated above, the unit dosage form of the invention comprises in a particular form of embodiment of the same, a low dose of a steroidal estrogen in which the positions 6 and 7 of the steroidal skeleton are both a —CH₂— residue, namely a dose of 5-5000 μg.

In an interesting embodiment of the invention, the film matrix comprises 10-3000 μg of said steroidal estrogen, such as 25-1500 μg of a steroidal estrogen.

The steroidal estrogen in which the positions 6 and 7 of the steroidal skeleton are both a —CH₂— residue may be selected from the group consisting of ethinylestradiol, estradiol estrone, mestranol, estriol, estriol succinate, estrone sulfate, 17β-estradiol sulfate, 17α-estradiol sulfate, estradiol valerate including therapeutically acceptable derivates thereof. In highly preferred embodiments of the invention, the estrogen is ethinylestradiol or estradiol, in particular ethinylestradiol.

Considering estradiol as the selected steroidal estrogen of a particular form of embodiment of the invention, the amount comprised in the film matrix is about 25-400 μg, more particularly 30-300 μg of Estradiol. The unit dosage form may contain estradiol in an “ultra-low” amount, i.e. 25-60 μg of estradiol, such as 30-50 μg of estradiol, preferably 40-50 μg of estradiol, e.g. about 40 μg, about 45 μg or about 50 μg of estradiol. The unit dosage form may also contain estradiol in a “very low” amount i.e. >60-200 μg of estradiol, such as 70-160 μg of estradiol, preferably 80-150 μg of estradiol, such as about 80 μg, 85 μg, 90 μg, 100 μg, 115 μg; 120 μg, 150 μg, or about 160 μg of estradiol.

If ethinylestradiol is incorporated in the unit dosage form according to this particular embodiment of the invention, the unit dosage form typically contains 10-30 μg of ethinylestradiol, such as about 15 μg or about 20 μg of ethinylestradiol.

Specific examples of doses of an 8β- or 9a-substituted estra-1,3,5(10)-triene as defined above, particularly a compound chosen from the group comprising:
9a-Vinyl-estra-1,3,5(10)-triene-3,16a-diol,
17β-Fluoro-9a-Vinyl-estra-1,3,5(10)-triene-3,16a-diol,
18a-Homo-9a-vinyl-estra-1,3,5(10)-triene-3,16a-diol,
16a-Fluoro-8β-vinyl-estra-1,3,5(10)-triene-3,17a-diol,
16a-Fluoro-8β-vinyl-estra-1,3,5(10)-triene-3,17β-diol,
16β-Fluoro-8β-vinyl-estra-1,3,5(10)-triene-3,17β-diol,
8β-Vinyl-estra-1,3,5(10)-triene-3,17β-diol, or derivatives thereof,
   more particularly for 17β-Fluoro-9a-vinyl-estra-1,3,5(10)-triene-3,16a-diol, or derivatives thereof, which may be comprised in the film matrix includes doses of about 10, 12.5, 15, 20, 25, 30, 40, 50, 60, 62.5, 70, 80, 90, 100, 120, 150, 180, 200, 250, 270, 300, 350, 360, 400, 450, 500, 540, 600, 625, 700, 800, 875, 900, 1000, 1100, 1250, 1500, 1750, 2000, 2500 or 3000 μg of ER-β selective agonist. Particular examples of doses of ER-β selective agonist which may be comprised in the film matrix are doses of about 15, 20, 25, 30, 40, 50, 60, 62.5, 70, 75, 80, 90, 100, 120, 150, 180, 200, 250, 270, 300, 500, 625, 875 or 1000 μg of ER-β selective agonist.

The above-mentioned doses preferably correspond to the daily dose. It should be understood that the above-mentioned doses are indicated with respect to estradiol ethinylestradiol or a ERβ selective agonist as defined above which is not esterified in position 3 or 7 of the steroidal skeleton. If a pharmaceutically acceptable ester of said active ingredients, is employed it will be understood that a dose which is therapeutically equivalent to the stated dose of the not esterified said active ingredients should be used. It is routine for those skilled in the art to determine pharmacologically/therapeutically equivalent doses of such other forms when the effective dose of the said active ingredient is known.

Stated differently, if a pharmaceutically acceptable ester of estradiol ethinylestradiol or a ERβ selective agonist as defined above, is employed it will be understood that a dose which is equimolar to the stated dose of the not esterified active ingredient should be used, provided that the absorption of the not esterified active ingredient and the derivative thereof is the same, cf. below. Thus, a “therapeutically equivalent amount of the Active Ingredient (AI) derivative X” can be calculated by the following formula:

Dose_{AI}×(MW_{AI derivative X}/MW_{AI})

where MW indicates the molecular weight of the active ingredient in question. It will be understood that all of the above-indicated intervals and doses of an estrogen as active ingredient should be converted to the corresponding intervals and doses (using the above formula) if the estrogen is used in its as a derivative. It will be understood, however, that the above formula can only be applied if the bioavailability and the Area Under the Curve (AUC) are identical for the estrogen and the derivative in question. Thus, if the absorption of the estrogen derivative in question differs from the absorption of the estrogen as such, the amount of the estrogen derivative required to achieve the plasma level of a given dose of the estrogen agonist is decisive for determining the therapeutically equivalent amount.

The paper of Timmer and Geurts provides guidance of how equivalent doses may be determined in the case of an estrogen (see “Bioequivalence assessment of three different estradiol formulations in postmenopausal women in an open, randomized, single-dose, 3-way cross-over” in European Journal of Drug Metabolism and Pharmacokinetics, 24(1):47-53, 1999).

According to a further form of embodiment of the present invention the unit dosage form comprises a progestin as an active ingredient. The amount of progestin incorporated in the unit dosage form of the invention is, of course, also dependent on the potency of the selected progestin, but will generally be in the range of from 0.1-30% (w/w) calculated on the basis of the unit dosage form. Typically, the amount of progestin incorporated in the unit dosage form of the invention is 0.1-25% (w/w), such as 0.2-20% (w/w).

More particularly, the unit dosage form may comprise desogestrel in an amount from 0.05-0.5 mg, preferably from 0.075-0.25 mg, such as 0.1 mg, 0.125 mg or 0.15 mg; ethynodiol diacetate in an amount from 0.25-2 mg, preferably 0.75-1.5 mg, such as 1 mg; levo-norgestrel in an amount from 0.025-0.3 mg, preferably from 0.075-0.25 mg, such as 0.1 mg or 0.15 mg; norethindrone (norethisterone) in an amount from 0.2-1.5 mg, preferably 0.3-1.25 mg, such as 0.4 mg, 0.5 mg or 1 mg; norethindrone (norethisterone) acetate in an amount from 0.5-2 mg, preferably 1-1.5 mg, such as 1 mg or 1.5 mg; norgestrel in an amount from 0.1-1 mg, preferably from 0.25-0.75 mg, such as 0.3 mg or 0.5 mg; norgestimate in an amount from 0.1-0.5 mg, preferably 0.15-0.3 mg, such as 0.18 mg, 0.215 mg or 0.25 mg; cyproterone acetate in an amount from 1-2 mg, preferably 2 mg; dienogest in an amount from 2-3 mg, preferably 2 mg; gestodene in an amount from 0.05-0.1 mg, preferably from 0.06-0.075 mg, such as 0.075 mg; and tibolone in an amount from 2-3 mg, such as 2.5 mg.

If the unit dosage form contains drospirenone as a progestinic component, the unit dosage form then typically contains 0.25-4 mg drospirenone, such as 1-4 mg drospirenone, e.g. about 1 mg, about 2 mg or about 3 mg drospirenone.

As discussed supra, at least one active ingredient may be complexed with a cyclodextrin and/or combined with an protective agent.

In one embodiment of the invention, the unit dosage form contains a active ingredient, preferably drospirenone, combined with a cationic polymethacrylate copolymer based on di-C₁₋₄-alkyl-amino-C₁₋₄-alkyl methacrylates and neutral methacrylic acid C₁₋₆-alkyl esters as protective agent. In a more preferred embodiment of the invention, the cationic polymethacrylate is a copolymer based on dimethylaminoethyl methacrylate and neutral methacrylic acid C₁₋₄-alkyl esters, such as a copolymer based on dimethyl-aminoethyl methacrylate, methacrylic acid methyl ester and methacrylic acid butyl ester. A particular preferred cationic polymethacrylate is poly(butyl methacrylate, (2-dimethyl aminoethyl)methacrylate, methyl methacrylate) 1:2:1. The cationic polymethacrylates mentioned above typically have an average molecular mass in the range of from 100,000 to 500,000 Da, such as an average molecular mass in the range of from 100,000 to 300,000 Da, e.g. an average molecular mass in the range of from 100,000 to 250,000 Da, preferably an average molecular mass in the range of from 100,000 to 200,000 such as an average molecular mass in the range of from 125,000 to 175,000 Da, e.g. an average molecular mass of about 150,000 Da. Such cationic polymethacrylates are available from Degussa, Germany, under the trade name Eudragit® E. In particular Eudragit® E 100 is preferred.

In another embodiment of the invention, the unit dosage form contains a active ingredient, preferably drospirenone, combined with a wax as protective agent. Examples of waxes include animal waxes, such as beewax, chinese wax, shellac wax, spermaceti wax and wool wax; vegetable waxes, such as carnauba wax, bayberry wax, candelilla wax, castor wax, esparto wax, ouricury wax, rice bran wax and soy wax; mineral waxes, such as ceresin wax, montan wax, ozocerite wax and peat wax; petroleum waxes, such as paraffin wax and microcrystalline wax; and synthetic waxes, such as polyethylene waxes, Fischer-Tropsch waxes, esterified and/or saponified waxes, substituted amide waxes and polymerised α-olefines. A particular preferred wax is carnauba wax.

If the active ingredient is combined with an protective agent, it is preferably provided in the form of particles comprising the active ingredient and the protective agent. Said particles should release as little active ingredient as possible in the mouth, while as much active ingredient as possible should be dissolved in the stomach and/or the intestine. This can be achieved, e.g., by embedding the active ingredient in the protective agent, for example in such a way that the active ingredient is present in a solid dispersion in the protective agent. This embodiment is particularly preferred when the protective agent is a cationic polymethacrylate. Alternatively, the active ingredient may be coated with the protective agent. This embodiment is particularly preferred when the protective agent is a wax.

The particles comprising the active ingredient and the protective agent have a d₉₀ particle size of ≦200 μm. In an interesting embodiment of the invention, the particles have a d₉₀ particle size of ≦175 μm, such as a d₉₀ particle size of 0.150 μm. As can be seen from the examples provided herein, the particle size of the particles comprising the active ingredient and the protecting agent is, at least to a certain extent, dependent on the applied protective agent. For example, if the protective agent is a cationic polymethacrylate copolymer, the particles comprising the active ingredient and the protective agent typically have a d₉₀ particle size in the range of from 50-200 μm, more typically in the range of from 50-150 μm, such as in the range of from 75-150 μm. Specific examples of d₉₀ particle sizes include values of about 50 μm, about 60 μm, about 70 μm, about 80 μm, about 90 μm, about 100 μm, about 110 μm, about 120 μm, about 130 μm, about 140 μm, and about 150 μm. Analogously, the d₅₀ particle size is typically in the range of from 5-80 μm, more typically in the range of from 10-75 μm, such as in the range of from 25-60 μm. Specific examples of d₅₀ particle sizes include values of about 5 μm, about 10 μm, about 20 μm, about 30 μm, about 40 μm, about 50 μm, about 60 μm, about 70 μm, and about 80 μm. On the other hand, if the protective agent is a wax, the particle size is significantly smaller. Thus, according to this embodiment of the invention, the particles comprising the active ingredient and the protective agent typically have a d₉₀ particle size in the range of from 0.1-40 μm, such as 0.2-30 μm, e.g. 0.4-25 μm, preferably 0.5-20 μm, such as 0.75-15 μm, e.g. 1-10 μm. Specific examples of d₉₀ particle sizes include values of about 1 μm, about 2 μm, about 3 μm, about 4 μm, about 5 μm, about 6 μm, about 7 μm, about 8 μm, about 9 μm, about 10 μm, about 15 μm, about 20 μm, and about 30 μm. Analogously, the d₅₀ particle size is typically in the range of from 0.1-10 μm, more typically in the range of from 0.5-7.5 μm, such as in the range of from 1-6 μm. Specific examples of d₅₀ particle sizes include values of about 0.5 μm, about 1 μm, about 2 μm, about 3 μm, about 4 μm, about 5 μm, about 6 μm, about 7 μm, about 8 μm, about 9 μm, and about 10 μm.

When used herein, the term “d₉₀ particle size” is intended to mean that the particle size distribution is so that at least 90% of the particles have a particle diameter of less than the specified value, calculated from the volume distribution curve under the presumption of spherical particles. In a similar way, the term “d₅₀ particle size” is intended to mean that the particle size distribution is so that at least 50% of the particles have a particle diameter of less than the specified value, calculated from the volume distribution curve under the presumption of spherical particles.

Therefore, it is important to note that whenever the terms “particle size”, “particle size distribution”, “particle diameter”, “d₉₀”, “d₅₀”, etc., are used herein it should be understood that the specific values or ranges used in connection therewith are always meant to be determined from the volume distribution curve under the presumption of spherical particles. The particle size distribution may be determined by various techniques, e.g. laser diffraction, and will be known to the person skilled in the art. The particles may be spherical, substantially spherical, or non-spherical, such as irregularly shaped particles or ellipsoidally shaped particles. Ellipsoidally shaped particles or ellipsoids are desirable because of their ability to maintain uniformity in the film forming matrix as they tend to settle to a lesser degree as compared to spherical particles. The particle size distribution of the particles comprising the active ingredient and the protective agent, when incorporated in the wafer, may be determined by dissolving the film forming matrix, separation of the protected particles, and drying the protected particles. The particle size distribution of the resulting particles may be determined as described above, e.g. by laser diffraction.

The unit dosage form of the invention is most preferably in the form of a thin film, which dissolves fast mainly due to the large surface area of the film, which wets quickly when exposed to the moist oral environment. Contrary to fast-dissolving tablets, which are usually soft, friable and/or brittle, the film is solid and strong, but still flexible. As indicated above, the film is thin and can be carried in the patient's pocket, wallet or pocket book.

The film may be applied under or on the tongue, to the upper palatine, to the inner cheeks or any oral mucosal tissue, of the female mammal. The film may be rectangular, oval, circular, or, if desired, a specific shape, cut to the shape of the tongue, the palatine or the inner cheeks, may be applied. The film is rapidly hydrated and will adhere onto the site of application. It then rapidly disintegrates and dissolves. An active ingredient can for example be released for oral mucosal absorption.

Concerning the dimensions of the unit dosage form of the invention, the water-soluble film forming matrix is formed into a dry film which typically has a thickness of less than 300 μm, in particular less than 250 μm, preferably less then 200 μm, such as less than 150 μm. More preferably, the thickness is less than 125 μm, such as less than 100 μm. Stated differently, the thickness is typically in the range of from 10-300 μm, in particular in the range of from 15-250 μm, preferably in the range of from 20-200 μm, such as in the range of from 25-150 μm. More preferably, the thickness is in the range of from 25-125 μm, such as in the range of from 25-100 μm, e.g. in the range of from 30-90 μm, in particular in the range of from 40-80 μm. Specific, and preferred, examples include thicknesses of about 30 μm, about 40 μm, about 50 μm, about 60 μm, about 70 μm, about 80 μm, about 90 μm or about 100 μm. Specific, and particularly preferred, examples include thicknesses of about 40 μm, about 50 μm, about 60 μm, about 70 μm or about 80 μm.

The surface dimension (surface area) of the film matrix is typically in the range of from 2-10 cm², such as in the range of from 3-9 cm², e.g. in the range of from 3-8 cm², more preferably in the range of from 3-7 cm², in particular in the range of from 4-6 cm². Specific, and preferred, examples of the surface area include surface areas of about 2, 3, 3.5, 4, 4.5, 5, 5.5, 6, 6.5 or 7 cm².

The total weight of the film matrix will typically be in the range of from 5-200 mg, such as in the range of from 5-150 mg, e.g. in the range of from 10-100 mg. More preferably, the total weight of the film matrix is in the range of from 10-75 mg, such as in the range of from 10-55 mg. Examples of the weight of the film matrix include weights of about 15 mg, about 20 mg, about 25 mg, about 30 mg, about 40 mg, about 50 mg or about 55 mg.

In an interesting embodiment of the invention, the unit dosage form further comprises an absorption enhancer. Absorption enhancers have demonstrated their effectiveness in delivering e.g. high molecular weight drugs, such as peptides, that generally exhibit low buccal absorption rates. Such absorption enhancers may act by a number of mechanisms, such a increasing the fluidity of the cell membrane, extracting inter/intracellular lipids, altering cellular proteins or altering surface mucin. The most commonly used absorption enhancers include azone, fatty acids, bile salts and surfactants, such as sodium dodecyl sulfate. Specific examples of absorption enhancers include, but are not limited to, 2,3-lauryl ether, aprotinin, azone, benzalkonium chloride, cetylpyridinium chloride, cetyltrimethyl ammonium bromide, cyclodextrin, dextran sulfate, glycol, lauric acid, lysophosphatidylcholine, menthol, phosphatidylcholine, polyoxyethylene, polysorbate 80, polyoxyethylene, phosphatidylcholine, sodium EDTA, sodium glycocholate, sodium glycodeoxycholate, sodium lauryl sulfate, sodium dodecyl sulfate, sodium salicylate, sodium taurocholate and sodium taurodeoxycholate, sulfoxides. The absorption enhancer is typically incorporated in the film matrix in an amount corresponding to 0.1-50% by weight of the film matrix, such as 1-20% by weight of the film matrix, e.g. 1-10% by weight of the film matrix. The absorption enhancer is typically comprised in the film matrix, i.e. the absorption enhancer is typically dissolved or dispersed in the film matrix. Preferably, no absorption enhancer is comprised.

In addition to the water-soluble matrix polymer and an active ingredient (and optionally one or more absorption enhancer(s)), the unit dosage form of the invention may include a variety of various auxiliary components, such as taste-masking agents; organoleptic agents, such as sweeteners and flavours, anti- and de-foaming agents; plasticizing agents; surfactants; emulsifying agents; thickening agents; binding agents; cooling agents; saliva-stimulating agents, such as menthol; antimicrobial agents; colorants; etc. Such various auxiliary components are comprised in the film matrix and is typically dissolved or dispersed in the film matrix.

Suitable sweeteners include both natural and artificial sweeteners. Specific examples of suitable sweeteners include, e.g.:

a) water-soluble sweetening agents such as sugar alcohols, monosaccharides, disaccharides, oligosaccharides and polysaccharides such as maltit, xylit, mannit, sorbit, xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochaicones, monellin, steviosides, and glycyrrhizin;
b) water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free add form of saccharin, and the like;
c) dipeptide-based sweeteners, such as L-aspartic acid derived sweeteners, such as L-aspartyl-L-phenylalanine methyl ester (aspartame), L-alpha-aspartyl-N-(2, 2,4,4 5 tetramethyl-3-thietanyl)-D-alaninamide hydrate, methyl esters of L-aspartyl-L phenylglycerin and L-aspartyl-L-2,5, dihydrophenylglycine, L-aspartyl-2,5-dihydro-L phenylalanine, L-aspartyl-L-(1-cyclohexyen)-alanine, and the like;
d) water-soluble sweeteners derived from naturally occurring water-soluble sweeteners, such as a chlorinated derivatives of ordinary sugar (sucrose), known, for example, under the product description of Sucralose®; and
e) protein-based sweeteners such as thaurnatoccous danielli (Thaurnatin I and II).

In general, an effective amount of sweetener is utilised to provide the level of sweetness desired for a particular composition, and this amount will vary with the sweetener selected. This amount will normally be from about 0.01% to about 20% by weight, preferably from about 0.05% to about 10% by weight, of the film matrix. These amounts may be used to achieve a desired level of sweetness independent from the flavour level achieved from any optional flavour oils used:

Useful flavours (or flavouring agents) include natural and artificial flavours. These flavourings may be chosen from synthetic flavour oils and flavouring aromatics, and/or oils, oleo resins and extracts derived from plants, leaves, flowers, fruits and so forth, and combinations thereof. Non-limiting examples of flavour oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavours such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavourings can be used individually or in combination. Commonly used flavours include mints such as peppermint, artificial vanilla, cinnamon derivatives, and various fruit flavours, whether employed individually or in combination. Flavourings such as aldehydes and esters including cinnamylacetate, cinnamaldehyde, citral, diethylacetal, dihydrocarvyl acetate, eugenyl formate, p-methylanisole, and the like may also be used. Further examples of aldehyde flavourings include, but are not limited to acetaldehyde (apple); benzaldehyde (cherry, almond); cinnamicaldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavours); butyraldehyde (butter, cheese); valeraldehyde (butter, cheese); citronellal (modified, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2-dimethyloctanal (greenfruit); and 2-dodecenal (citrus, mandarin); cherry; grape; essential oils, like menthol; mixtures thereof; and the like.

The amount of flavouring employed is normally a matter of preference, subject to such factors as flavour type, individual flavour, and strength desired. The amount may be varied in order to obtain the result desired in the final product. Such variations are within the capabilities of those skilled in the art without the need for undue experimentation. In general, amounts from about 0.01% to about 10% by weight of the film matrix are employed.

As discussed above, the unit dosage form may also include an anti-foaming and/or de-foaming agent, such as simethicone, which is a combination of a polymethylsiloxane and silicon dioxide. Simethicone acts as either an anti-foaming or de-foaming agent which reduces or eliminates air from the film composition. Anti-foaming agents will aid in preventing the introduction of air into the composition, while de-foaming agents will aid removing air from the composition. Preferably, no anti-foaming or de-foaming agent is comprised.

The unit dosage form may be prepared and adhered to a second layer, i.e. a support or backing layer (liner) from which it is removed prior to use, i.e. before being introduced into the oral cavity. Preferably, the support or backing material is not water-soluble and may preferably consist of polyethylene-terephthalate, or other suitable materials well known to the skilled person.

If an adhesive is used, it should preferably be a food grade adhesive that is not ingestible and does not alter the properties of the active ingredient(s).

In a particular form of embodiment of the invention, a steroidal estrogen as defined above, more particular a ERβ selective agonist, or a derivative thereof, is the only or sole therapeutically active drug substance present in the unit dosage form.

In another embodiment of the invention, the unit dosage form of the invention comprises more than one drug substance, in particular at least an estrogen and at least a progestin. Particularly preferred progestins are 16,17-carbolactone derivatives (for example drospirenone), and levonorgestrel, dienogest, gestodene, and cyproterone acetate. Other specific examples of progestins which can be comprised in the wafer according to the invention were explicitly mentioned above.

At least one active drug substance is comprised in the film matrix.

While the present disclosure is mainly concerned with wafers containing a ERβ selective agonist, or derivatives thereof, it is contemplated that the invention can also be practiced with other compounds exhibiting estrogenic activity, such as estradiol, ethinylestradiol, estrone, estradiol, estradiol valerate, 17β-estradiol sulfate, 17α-estradiol sulphate, mestranol, estriol, estriol succinate, including therapeutically acceptable derivates or compounds exhibiting progestinic activity such as levonorgestrel, norgestrel, norethindrone (norethisterone), dienogest, norethindrone (norethisterone) acetate, ethynodiol diacetate, norethynodrel, allylestrenol, lynestrenol, norgestrienone, ethisterone, promegestone, desogestrel, 3-keto-desogestrel, norgestimate, gestodene including therapeutically acceptable derivates. In general the present invention can be practiced with an active ingredient being a steroid in which the positions 6 and 7 of the steroidal skeleton are both a —CH₂— residue.

Accordingly, in one aspect, the present invention relates to a unit dosage form comprising a thin water-soluble film matrix, wherein said film matrix comprises
a) a polyvinyl alcohol-polyethylene glycol graft copolymer (PVA-PEG graft co-polymer) as a water-soluble matrix polymer;
b) a 8β- or 9a-substituted estra-1,3,5(10)-triene as a Estrogen Receptor beta (ERβ) selective agonist, in particular a ERbeta selective agonist chosen from the compounds:
9a-Vinyl-estra-1,3,5(10)-triene-3,16a-diol,
17β-Fluoro-9a-Vinyl-estra-1,3,5(10)-triene-3,16a-diol,
18a-Homo-9a-vinyl-estra-1,3,5(10)-triene-3,16a-diol,
16a-Fluoro-8β-vinyl-estra-1,3,5(10)-triene-3,17β-diol,
16β-Fluoro-8β-vinyl-estra-1,3,5(10)-triene-3,17β-diol,
163-Fluoro-8β-vinyl-estra-1,3,5(10)-triene-3,173-diol,
8β-Vinyl-estra-1,3,5(10)-triene-3,173-diol, or derivatives thereof; and
   said film matrix has a thickness of less than 300 μm.

### Therapeutic Use and Administration

It will be understood that the unit dosage form of the invention is administered intraorally, i.e. the unit dosage form is administered to the oral cavity and the active drug substance is subsequently absorbed via one or more of the oral mucosae. Thus, the active drug substance is suitable for lingual administration, sublingual administration, buccal administration and palatal administration.

Accordingly, in another aspect, the present invention relates to a unit dosage form of the invention for use as a medicament.

In yet another aspect, the present invention relates to a unit dosage form to be used in Hormone Replacement Therapy (HRT) and in particular for treating, alleviating or preventing a physical condition in a female mammal caused by insufficient endogenous levels of estrogen. Examples of such physical conditions include, but is not limited to, osteoporosis, headaches, nausea, depression, vasomotor symptoms, symptoms of urogenital atrophy, decrease in bone mineral density, and increased risk or incidence of bone fracture.

The drug delivery systems in the form of thin water-soluble films (wafers) according to the present invention can be favourable used for contraception.

The unit dosage forms of the present invention have a considerable higher bioavailability than orally administered tablets particularly with reference to steroidal estrogens.

Thus, a bioavailability of more than 30% will typically be achieved for such an estrogen. More particularly, a bioavailability in the range of from 30-100%, such as 40-90% will typically be achieved. In an interesting embodiment of the invention, a bioavailability of more than 50%, particularly more than 60% is achieved.

This, in turn, has the consequence that therapeutic effective serum levels of an active ingredient, can be achieved although a significantly lower dose of said active ingredient is administered as compared to oral administration. This is for example the case when an ERβ selective agonist as steroidal estrogen according to the invention is the active ingredient.

Evidently, the achieved bioavailability as well as the serum level of the active ingredient will be dependent on the actual design of the unit dosage form as well as the drug load and the applied active ingredient.

### Manufacture

The drug delivery system, equally defined as unit dosage form according to the present invention, may be prepared by standard methods well known to the pharmaceutical technologist.

Typically, a drug solution is prepared by dissolving the active ingredient, or a derivative thereof, in an appropriate solvent. The solvent is preferably a relatively volatile solvent, such as an alcohol, in particular ethanol. A matrix polymer solution comprising a PVA-PEG co-polymer is then prepared by adding the water-soluble matrix polymer to a suitable solvent, such as water or alcohol or a mixture of an alcohol and water. Preferably, the solvent is an ethanol/water mixture. As will be understood, the time and conditions needed to dissolve the water-soluble matrix polymer will depend on the polymer and the solvent used. Thus, in some cases the water-soluble matrix polymer may dissolve easily at room temperature and with only gentle stirring, while in other cases it will be necessary to apply heat and vigorous stirring to the system. In a typical embodiment, the mixture is stirred for 1-4 hours, preferably for about 2 hours, or until a solution is obtained. The solution is typically stirred at a temperature of 60-80° C., such as about 70° C. After cooling to room temperature, the drug solution is poured into the matrix polymer solution and mixed thoroughly. The resulting solution (coating solution) can be used for coating immediately or within a few days. The various amounts of solvent, matrix polymer, etc. are adjusted to reach a solid content of the coating solution of about 5-50% by weight, preferably 10-40% by weight, in particular 20-35% by weight.

In an alternative embodiment, the coating solution may be prepared directly by adding the active ingredient, or a derivative thereof, to an appropriate solvent, preferably an alcohol, in particular ethanol, followed by addition of water and subsequent addition of the matrix polymer. The mixture is then processed as described above until a solution is obtained. The resulting solution (coating solution) can be used for coating immediately or within a few days. The various amounts of solvent, matrix polymer, etc. are adjusted to reach a solid content of the coating solution of about 5-50% by weight, preferably 10-40% by weight, in particular 20-35% by weight.

In an alternative embodiment, the coating solution may be prepared by directly adding the active ingredient, or a derivative thereof, to an appropriate polymer solution and dissolving the drug in it. In this case the polymer solution was prepared beforehand by dissolving the polymer in the solvent/water mixture according the above described process. After dissolution of the active ingredient in the polymer solution, the resulting solution (coating solution) can be used for coating immediately or within a few days. The various amounts of solvent, matrix polymer, etc. are adjusted to reach a solid content of the coating solution of about 5-50% by weight, preferably 10-40% by weight, in particular 20-35% by weight.

Other excipients, auxiliary components and/or active drug substances may be added during any of the above mentioned steps.

If needed, the coating solution is degassed before being spread out on a suitable support or release liner (liner). Examples of suitable liners include, but are not limited to polyethylene-terephthalate (PET) liners, such as Perlasic® LF75 (available from Perlen Converting), Loparex® LF2000 (available from Loparex BV) and Scotchpack® 9742 (available from 3M Drug delivery Systems). In one embodiment of the invention, the coating solution is spread out with the aid of a spreading box onto a suitable liner and dried for 12-24 hours at room temperature. A thin transparent film of 30-100 μm thickness, preferably 40-80 μm thickness is then produced, which is subsequently cut into pieces of the desired size and shape. Alternatively, the coating solution is coated as a thin film onto a suitable liner and in-line dried using an automated coating and drying equipment (e.g. by Coatema Coating Machinery GmbH, Dormagen, Germany) using a drying temperature of 40-120° C. A thin transparent film is then produced, which is subsequently cut into pieces of the desired size and shape.

According to the above manufacturing procedures, both drug delivery systems comprising a PVA-PEG co-polymer according to the invention and drug delivery systems without said polymer were prepared as described in the examples below.

### A. Wafers According to the Invention and Preparation Thereof.

The following examples (1-4) of unit dosage forms comprising a thin water-soluble film matrix (wafer), as well as their method of preparation, are intended as a illustrative not limiting examples of unit dosage forms and their method of preparation according to the invention. For the following examples of wafers comprising a ERβ selective agonist as an active ingredient, the compound 1713-Fluoro-9a-vinyl-estra-1,3,5(10)-triene-3,16a-diol was used.

The wafers provided below should be considered as non-limiting examples of unit dosage forms according to the invention comprising an active ingredient being a steroid in which the positions 6 and 7 of the steroidal skeleton are both a —CH₂— group.

In the present application, any amount given in percentage (%) should be intended as percentage by weight (% w/w) if not differently specified.

### Example 1

### Manufacturing of Wafers

### Preparation of the Coating Solution

### Option A

A drug solution containing 0.725 g ERβ selective agonist is prepared by dissolving the drug in 236.7 g ethanol (96%) under stirring. A polymer solution is prepared by strewing 28.9.275 g PVA-PEG-Copolymer (Macrogol poly(vinyl alcohol) grafted copolymer) onto 710 g of a water/ethanol mixture of the ratio 2:1 or 289.275 g of Hydroxypropylcellulose (HPC) or Hydroxypropyl methylcellulose (HPMC) onto 710 g of a water/ethanol mixture of the ratio 1:2. The polymer dissolves after stirring for 1-2 hours at 70° C. After cooling to room temperature, the drug solution is poured into the polymer solution and mixed thoroughly. The resulting solution (coating solution) can be used for coating immediately or within a few days.

### Option B

A coating solution is prepared by dissolving 0.725 g ERβ selective agonist in ethanol (96%) under stirring. After admixing with water, 289.275 g of the respective polymer is added. 236.7 g ethanol and 473.3 g water are used in case of PVA-PEG-Copolymer (Macrogol poly(vinyl alcohol) grafted copolymer) as the matrix polymer. 473.3 g ethanol and 236.7 g water are used in case of Hydroxypropylcellulose (HPC) or Hydroxypropyl methylcellulose (HPMC) as the matrix polymer. The polymer is added and dissolves after stirring for 2 hours at 70° C. The resulting solution (coating solution) can be used for coating immediately or within a few days.

### Preparation of Wafers

### Option 1

The coating solution is degassed and spread out with the aid of a spreading box onto a polyethylene-terephthalate (PET) liner (e.g. Scotchpak® 9742 or Perlasic®LF75) and dried for 24 hours at room temperature. A thin film which is about 40-70 μm thick is produced. Wafers are obtained by punching out samples of 2-7 cm² size.

### Option 2

The coating solution is degassed and coated as a thin film onto a polyethylene-terephthalate (PET) liner (e.g. Scotchpak® 9742 or Perlasic® LF75) and in-line dried using an automated coating and drying equipment (Coatema Coating Machinery GmbH, Dormagen, Germany). A drying temperature of 40-120° C. is applied. A thin film which is about 40-70 μm thick is produced. Wafers are obtained by punching out samples of 2-7 cm² size.

Using the above-mentioned manufacturing methods, wafers having the following composition were prepared (examples 1a-f, 2a-b, 3a-b, and 4):

### Drug Delivery System (Wafer) Comprising PVA-PEG-Copolymer According to the Invention:

### Example 1a

### ERβ Selective Agonist Wafer, 25 μg (with PVA-PEG-Copolymer Matrix), 2 cm², Active Ingredient Concentration 0.25%

| Name of ingredient | Quantity | Function |
|---|---|---|
| Active ingredients | | |
| ERβ selective agonist | 0.025 mg | Active ingredient |
| Other ingredients | | |
| PVA-PEG-Copolymer | 9.975 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 10.000 mg | |

*evaporates during manufacturing

### Example 1b

### ERβ Selective Agonist Wafer, 150 μg (with PVA-PEG-Copolymer Matrix), 3 cm², Active Ingredient Concentration 1%

| Name of ingredient | Quantity | Function |
|---|---|---|
| Active ingredients | | |
| ERβ selective agonist | 0.150 mg | Active ingredient |
| Other ingredients | | |
| PVA-PEG-Copolymer | 14.850 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 15.000 mg | |

*evaporates during manufacturing

### Example 1c

### ERβ Selective Agonist Wafer, 62.5 μg (with PVA-PEG-Copolymer Matrix), 5 cm², Active Ingredient Concentration 0.25%

| Name of ingredient | Quantity | Function |
|---|---|---|
| Active ingredients | | |
| ERβ selective agonist | 0.0625 mg | Active ingredient |
| Other ingredients | | |
| PVA-PEG-Copolymer | 24.9375 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 25.000 mg | |

*evaporates during manufacturing

### Example 1d

### ERβ Selective Agonist Wafer, 250 μg (with PVA-PEG-Copolymer Matrix), 5 cm², Active Ingredient Concentration 1%

| Name of ingredient | Quantity | Function |
|---|---|---|
| Active ingredients | | |
| ERβ selective agonist | 0.250 mg | Active ingredient |
| Other ingredients | | |
| PVA-PEG-Copolymer | 24.750 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 25.000 mg | |

*evaporates during manufacturing

### Example 1e

### ERβ Selective Agonist Wafer, 625 μg (with PVA-PEG-Copolymer Matrix), 5 cm², Active Ingredient Concentration 2.5%

| Name of ingredient | Quantity | Function |
|---|---|---|
| Active ingredients | | |
| ERβ selective agonist | 0.625 mg | Active ingredient |
| Other ingredients | | |
| PVA-PEG-Copolymer | 24.375 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 25.000 mg | |

*evaporates during manufacturing

### Example 1f

### ERβ Selective Agonist Wafer, 875 μg (with PVA-PEG-Copolymer Matrix), 7 cm², Active Ingredient Concentration 2.5%

| Name of ingredient | Quantity | Function |
|---|---|---|
| Active ingredients | | |
| ERβ selective agonist | 0.875 mg | Active ingredient |
| Other ingredients | | |
| PVA-PEG-Copolymer | 34.125 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 35.000 mg | |

*evaporates during manufacturing

As will be understood, analogous wafers which contain other amounts of a ER-β selective agonist, can easily be manufactured using the procedures described herein.

### Example 2a

### Estradiol, 150 μg (with PVA-PEG-Copolymer Matrix), 5 cm², Active Ingredient Concentration 0.6%

| Name of ingredient | Quantity | Function |
|---|---|---|
| Active ingredients | | |
| Estradiol hemihydrate | 0.155 mg | Active ingredient |
| (~0.150 mg estradiol) | | |
| Other ingredients | | |
| PVA-PEG-Copolymer | 24.845 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 25.000 mg | |

*evaporates during manufacturing

### Example 2b

### Estradiol, 80 μg (with PVA-PEG-Copolymer Matrix), 5 cm² Active Ingredient Concentration 0.3%

| Name of ingredient | Quantity | Function |
|---|---|---|
| Active ingredients | | |
| Estradiol hemihydrate | 0.083 mg | Active ingredient |
| (~0.080 mg estradiol) | | |
| Other ingredients | | |
| PVA-PEG-Copolymer | 23.917 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 24.000 mg | |

*evaporates during manufacturing

### Example 3a

### Ethinyl Estradiol, 15 μg (with PVA-PEG-Copolymer Matrix), 5 cm² Active Ingredient Concentration 0.06%

| Name of ingredient | Quantity | Function |
|---|---|---|
| Active ingredients | | |
| Ethinyl estradiol | 0.015 mg | Active ingredient |
| Other ingredients | | |
| PVA-PEG-Copolymer | 24.985 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 25.000 mg | |

*evaporates during manufacturing

### Example 3b

### Ethinyl Estradiol, 20 μg (with PVA-PEG-Copolymer Matrix), 5 cm² Active Ingredient Concentration 0.08%

| Name of ingredient | Quantity | Function |
|---|---|---|
| Active ingredients | | |
| Ethinyl estradiol | 0.020 mg | Active ingredient |
| Other ingredients | | |
| PVA-PEG-Copolymer | 24.980 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 25.000 mg | |

*evaporates during manufacturing

### Example 4

### Levonorgestrel, 125 μg (with PVA-PEG-Copolymer Matrix), 5 cm² Active Ingredient Concentration 0.5%

| Name of ingredient | Quantity | Function |
|---|---|---|
| Active ingredients | | |
| Levonorgestrel | 0.125 mg | Active ingredient |
| Other ingredients | | |
| PVA-PEG-Copolymer | 24.875 mg | Matrix polymer |
| (Macrogol poly(vinyl | | |
| alcohol) grafted copolymer) | | |
| Purified water* | q.s. | Process solvent |
| Ethanol 96%* | q.s. | Process solvent |
| Total mass: | 25.000 mg | |

*evaporates during manufacturing

As will be understood that analogous wafers which contain other amounts of an active ingredient being a steroid in which the positions 6 and 7 of the steroidal skeleton are both a —CH₂— can easily be manufactured using the procedures described herein.

PVA-PEG co-polymer used in the above compositions given as an illustrative example was Kollicoat® IR which comprises 75% polyvinyl alcohol units and 25% polyethylene glycol units.

Preparation of Wafers Comprising at Least an Active Ingredient Complexed with a Cyclodextrin, and/or Combined with a Protective Agent According to the Invention

Further examples (5-7) of unit dosage forms comprising a thin water-soluble film matrix (wafer) in which at least an active ingredient is complexed with a cyclodextrin, and/or combined with a protective agent, as well as their method of preparation, are reported below and are intended as a illustrative, not limiting examples of unit dosage forms according to the invention.

### Example 5

### Preparation of Particles Comprising Drospirenone as Active Ingredient And a Protective Agent

### Example 5a

### Drospirenone/Carnauba Wax

80 g of carnauba wax (Pharm. Grade) was dissolved in 1 kg of n-heptane at 60° C. in a 2 litre double-walled glass beaker while stirred at 400 rpm until a clear solution was obtained.

80 g of micronized drospirenone was added slowly to the solution to avoid clumping while the stirring rate was adjusted to 600 rμm. The mixture was cooled to 20° C. at a cooling rate of 20° C./hour to yield the drug containing microparticles coated with Carnauba wax.

The drospirenone-containing microparticles were filtrated using a cellulose acetate filter membrane and a glass filter unit. The microparticles were subsequently washed with 300 ml ethanol (96%) to remove n-heptane residues and non-encapsulated drospirenone. The filtered microparticles were transferred to a glass bowl and dried for 2 hours at 30° C. The resulting protected particles, wherein the drospirenone is coated with the protective agent, had a d₅₀ particle size of 2.2 μm and a d₉₀ particle size of 4.8 μm.

### Example 5b

### Drospirenone/Eudragit® 100 Prepared by Milling

20 g of drospirenone and 80 g of Eudragit® E 100 were dissolved in 200 ml of a mixture of ethanol and acetone 7+23 (w+w) in a 300 ml glass beaker while stirring at 200 rpm at room temperature for 1 hour. A clear solution was obtained.

The solution was then transferred into a siliconized pan. The solution was dried under ambient conditions in a hood for 3 days to remove the acetone. A sensual test was used to indicate the absence of acetone. The thus obtained stiff film had a thickness of a few millimeters and was manually broken into parts of about 10 cm². These parts were subsequently milled using a rotor mill (Retsch ultra centrifugation mill ZM200) under cooling with dry ice. The resulting protected particles, wherein the drospirenone is present in a solid dispersion in the protective agent, had a d₅₀ particle size of 20-50 μm and a d₉₀ particle size of 80-100 μm. The protected particles are stored protected from heat (e.g. in a refrigerator) until further use.

### Example 5c

### Drospirenone/Eudragit® E 100 Prepared by Spraydrying

20 g of drospirenone and 80 g of Eudragit® E 100 were dissolved in 200 ml of ethanol 96% in a 300 ml glass beaker while stirring at 200 rpm at room temperature for 1 hour. A clear solution was obtained.

The solution was spraydried at about 35° C. The resulting protected particles, wherein the drospirenone is present in a solid dispersion in the protective agent, had a d₅₀ particle size of 5-50 μm and a d₉₀ particle size of <100 μm. The protected particles are stored protected from heat (e.g. in a refrigerator) until further use.

### Example 6

### Preparation of a Coating Solutions Comprising an Estrogen in which the Positions 6 and 7 of the Steroidal Skeleton are both a —CH₂— Group and Particles Containing Drospirenone and a Protective Agent

### Example 6a

### Kollicoat° IR Matrix/Ethinylestradiol/Drospirenone Particles

43.985 g of Kollicoat® IR was dissolved in 78 ml of purified water in a glass beaker at 60-80° C. while stirring at 100 rpm for 2 hours. A clear solution was obtained (polymer solution). After cooling, the evaporated water was replaced.

15 mg of ethinylestradiol was dissolved in 2 ml of ethanol (96%) with stirring under ambient conditions (ethanol solution).

6 g of the particles prepared in Example 5a were dispersed in a mixture of 8 ml ethanol and 12 ml water and then added to the polymer solution while stirring. The stirring speed and time were adjusted to obtain a homogenous dispersion (coating solution). Subsequently, the ethanol solution was added (coating solution).

### Example 6b

### Kollicoat® IR Matrix/Estradiol/Drospirenone Particles

43.907 g of Kollicoat® IR was dissolved in 78 ml of purified water in a glass beaker at 60-80° C. while stirring at 100 rpm for 2 hours. A clear solution was obtained (polymer solution). After cooling, the evaporated water was replaced.

93 mg estradiol hemihydrate was dissolved in 2 ml of ethanol (96%) with stirring under ambient conditions (ethanol solution).

6 g of the particles prepared in Example 5a were dispersed in a mixture of 8 ml ethanol and 12 ml water and then added to the polymer solution while stirring. The stirring speed and time were adjusted to obtain a homogenous dispersion (coating solution). Subsequently, the ethanol solution was added (coating solution).

### Example 7

### Preparation of Wafers

The preparation of wafers from the coating solutions according to example 6 is performed according to Example 1, Option 2 (see above):

### Drug Delivery System (Wafer) Comprising PVA-PEG-Copolymer and at Least an Active Ingredient Complexed with a Cyclodextrin and/or Combined with a Protective Agent

The following examples (7j, 7k, 7l, 7o, and 7p) were obtained by preparing particles according to example 5, followed by the preparation of coating solutions according to examples 6 and the preparation of wafers according to example 1, Option 2 as described above.

### Example 7j

### Ethinylestradiol, 15 μg and Drospirenone, 3 mg (as Protected Particles) (with PVA-PEG-Copolymer Matrix), 7 cm²

| Ingredient | Amount | | Function |
|---|---|---|---|
| Ethinylestradiol | 0.015 | mg | Active ingredient |
| (unprotected) | | | |
| Drospirenone | 3.0 | mg | Active ingredient |
| Eudragit ® E 100 | 12.0 | mg | Protective agent for Drospirenone |
| Kollicoat ® IR | 34.985 | mg | Matrix polymer |
| Total | 50 | mg | |

### Example 7k

### Ethinylestradiol, 15 μg and Drospirenone, 3 mg (as Protected Particles) (with PVA-PEG-Copolymer Matrix), 7 cm²

| Ingredient | Amount | | Function |
|---|---|---|---|
| Ethinylestradiol | 0.015 | mg | Active ingredient |
| (unprotected) | | | |
| Drospirenone | 3.0 | mg | Active ingredient |
| Carnauba wax | 3.0 | mg | Protective agent for Drospirenone |
| Kollicoat ® IR | 43.985 | mg | Matrix polymer |
| Total | 50 | mg | |

### Example 7l

### Ethinylestradiol, 15 μg (Complexed with Beta-Cyclodextrin) and Drospirenone, 3 mg (as Protected Particles) (with PVA-PEG-Copolymer Matrix), 7 cm²

| Ingredient | Amount | | Function |
|---|---|---|---|
| Ethinylestradiol betadex* | 0.130 | mg | Active ingredient |
| Drospirenone | 3.0 | mg | Active ingredient |
| Carnauba wax | 3.0 | mg | Protective agent for Drospirenone |
| Kollicoat ® IR | 43.87 | mg | Matrix polymer |
| Total | 50 | mg | |

*as beta-cyclodextrin clathrate; corresponds to 0.015 mg ethinylestradiol; thus it is to be understood that ethinyl estradiol is not combined with a protective agent

### Example 7o

### Estradiol, 150 μg and Drospirenone, 3 mg (as Protected Particles) (with PVA-PEG-Copolymer Matrix), 7 cm²

| Ingredient | Amount | | Function |
|---|---|---|---|
| Estradiol hemihydrate* | 0.155 | mg | Active ingredient |
| (unprotected) | | | |
| Drospirenone | 3.0 | mg | Active ingredient |
| Eudragit ® E 100 | 12.0 | mg | Protective agent for Drospirenone |
| Kollicoat ® IR | 34.845 | mg | Matrix polymer |
| Total | 50 | mg | |

*Corresponds to 0.150 mg estradiol

### Example 7p

### Estradiol, 120 μg and Drospirenone, 3 mg (as Protected Particles) (with PVA-PEG-Copolymer Matrix), 7 cm²

| Ingredient | Amount | | Function |
|---|---|---|---|
| Estradiol hemihydrate* | 0.124 | mg | Active ingredient |
| (unprotected) | | | |
| Drospirenone | 3.0 | mg | Active ingredient |
| Carnauba wax | 3.0 | mg | Protective agent for Drospirenone |
| Kollicoat ® IR | 43.876 | mg | Matrix polymer |
| Total | 50 | mg | |

*Corresponds to 0.120 mg estradiol

### C. Drug Delivery System (Wafer) Without PVA-PEG-Copolymer for Comparative Tests:

The following examples (8a-f) of wafers without a PVA-PEG-copolymer were obtained preparing a coating solution according to Example 1, Option A or B and the following preparation of said wafer according to Example 1, Option 1 or 2 as described above.

### Example 8a

ERβ selective agonist wafer, 87.5 μg (with Hydroxypropyl Cellulose Matrix), 7 cm², Active Ingredient Concentration 0.25%

| Name of ingredient | Quantity | Function |
|---|---|---|
| Active ingredients | | |
| ERβ selective agonist | 0.0875 mg | Active ingredient |
| Other ingredients | | |
| Hydroxypropyl cellulose | 34.9125 mg | Matrix polymer |
| Purified water* | q.s. | Process solvent |
| Ethanol 96%* | q.s. | Process solvent |
| Total mass: | 35.000 mg | |

*evaporates during manufacturing

### Example 8b

ERβ Selective Agonist Wafer, 350 μg (with Hydroxypropyl Cellulose Matrix), 7 cm², Active Ingredient Concentration 1%

| Name of ingredient | Quantity | Function |
|---|---|---|
| Active ingredients | | |
| ERβ selective agonist | 0.350 mg | Active ingredient |
| Other ingredients | | |
| Hydroxypropyl cellulose | 34.650 mg | Matrix polymer |
| Purified water* | q.s. | Process solvent |
| Ethanol 96%* | q.s. | Process solvent |
| Total mass: | 35.000 mg | |

*evaporates during manufacturing

### Example 8c

### ERβ Selective Agonist Wafer, 875 μg (with Hydroxypropyl Cellulose Matrix), 7 cm², Active Ingredient Concentration 2.5%

| Name of ingredient | Quantity | Function |
|---|---|---|
| Active ingredients | | |
| ERβ selective agonist | 0.875 mg | Active ingredient |
| Other ingredients | | |
| Hydroxypropyl cellulose | 34.125 mg | Matrix polymer |
| Purified water* | q.s. | Process solvent |
| Ethanol 96%* | q.s. | Process solvent |
| Total mass: | 35.000 mg | |

*evaporates during manufacturing

### Example 8d

### ERS Selective Agonist Wafer, 875 μg (with Hydroxypropyl methylcellulose Matrix), 7 cm², Active Ingredient Concentration 2.5%

| Name of ingredient | Quantity | Function |
|---|---|---|
| Active ingredients | | |
| ERβ selective agonist | 0.875 mg | Active ingredient |
| Other ingredients | | |
| Hydroxypropyl | 34.125 mg | Matrix polymer |
| methylcellulose | | |
| Purified water* | q.s. | Process solvent |
| Ethanol 96%* | q.s. | Process solvent |
| Total mass: | 35.000 mg | |

*evaporates during manufacturing

### Example 8e

Additional formulations were prepared with other polymers, such as polyvinylpyrrolidone (PVP) or polyvinylalcohol (PVA) (grade: 4-88), with same procedure as described above.

In some formulations also additives, such as plasticizer (e.g. propylene glycol (PG), triethylcitrate (TEC)) or stabilizers (e.g. different grades of cyclodextrins (CD) such as gamma-Cyclodextrin), or antioxidants (e.g. buthylhydroxytoluol (BHT) or propylgallate) were added in the indicated amount.

### Example 8f

Placebo wafers were manufactured accordingly by dissolving polymers and additives in ethanol/water 2:1 to receive the coating solution. From this coating solutions, wafers were prepared according to example 1, Option 1 as described above.

### Stabilising Effect of the Drug Delivery System (Wafer) on the Active Ingredient

The stabilising effect of the drug delivery system according to the invention on the drug substance comprised in it, was investigated at ambient (room) and accelerated conditions (40° C./75% r.h.).

The drug substance comprised in the unit dosage form was in the range of 0.25-2.5% in weight.

The compound 17β-Fluoro-9a-vinyl-estra-1,3,5(10)-triene-3,16a-diol was used in the tested unit dosage forms and should be considered as a non-limiting example of a steroid in which the positions 6 and 7 of the steroidal skeleton are both a —CH₂—.

Although HPMC is the most commonly used film matrix polymer, several other polymers were investigated under the same conditions in particular (HPC (Klucel®EF); PVP (Kollidon® 30); and PVA (4-88).

In a HPMC wafers, at a drug concentration of 0.25% in the polymer matrix according to the example reported above, 21.8% of the drug was degraded after 1 month storage at accelerated conditions (40° C./75% r.h.).

Moreover, common stabilizing techniques (antioxidants, cyclodextrins (CD)) known to improve steroids stability were also tested. However, none of the known technique achieved an improved stability of the steroidal ERβ selective agonist 17β-Fluoro-9a-vinyl-estra-1,3,5(10)-triene-3,16a-diol.

Even cyclodextrins, usually providing a pronounced stabilizing effect on steroid hormones, revealed no effect on the stability of the above compound in an HPMC-matrix.

Surprisingly, the stability of the chosen steroid in was found to be superior in a wafer comprising a PVA-PEG-Copolymer.

Drug Content of ERβ Selective Agonist in Different Matrix Polymers with And without Stabilizers Before and after Open Storage (Drug Conc. in the Polymer Matrix: 0.25%) Compared to a Drug Content in a Unit Dosage Form According to the Present Invention Comprising PVA-Peg-Copolymer.

| | 0 month, | 1 month | loss |
|---|---|---|---|
| | % of label | 40° C./75% r.h., | in drug |
| Polymer + Additives | claim | % of label claim | content, % |
| HPMC | 95.8 | 74 | 21.8 |
| HPMC + gamma CD * | 95.5 | 73.6 | 21.9 |
| HPMC + Beta-CD ** | 93.1 | 70.8 | 22.3 |
| HPMC + HP-beta-CD ** | 96.5 | 71.2 | 25.3 |
| HPMC + Propylgallat (10%) | 94.6 | 73.2 | 21.4 |
| HPMC + BHT 0.01% | 100.3 | 78.4 | 21.9 |
| (+Propylenglycol 10%) | | | |
| HPC | 93.9 | 70 | 23.9 |
| PVP (+BHT 0.01%) | 85.5 | 65.2 | 20.3 |
| PVA (+BHT 0.01%) | 95.1 | 72.4 | 22.7 |
| PVA-PEG-Copolymer | 94.9 | 78.4 | 16.5 |

* ratio of active/CD 1:2
** ratio of active/CD 1:5

As clearly appears from the above results (0.25% drug conc. in the polymer matrix), most polymers revealed a loss of active about 20% after 1 month storage at accelerated conditions.

PVP revealed already an initially stronger degradation of the active compared to other polymers, even with the addition of stabilizers (e.g. Butylhydroxytoluol (BHT)).

However, surprisingly, a PVA-PEG-Copolymer according to the present invention revealed a lower extent of degradation (16.5% loss of active) compared to all other polymers (>20% loss of active) at a drug conc. of 0.25% in the polymer matrix.

This was also confirmed at higher drug concentration and longer storage periods as reported in the tables below.

Drug Content of ERβ Selective Agonist in Different Matrix Polymers with And without Stabilizers Before and after Open Storage (Drug Conc. in the Polymer Matrix: 1%) Compared to a Drug Content in a Unit Dosage Form According to the Present Invention Comprising PVA-Peg-Copolymer.

| | 0 month, | 3 months | loss |
|---|---|---|---|
| | % of label | room cond., | in drug |
| Polymer | claim | % of label claim | content, % |
| HPMC + Propylgallat (10%) | 95.6 | 92.9 | 3.6 |
| HPMC + BHT 0.01% | 102.0 | 96.8 | 5.2 |
| (+Propylenglycol 10%) | | | |
| HPC | 102.2 | 94.9 | 7.3 |
| PVP (+BHT 0.01%) | 88.2 | 83.1 | 5.1 |
| PVA (+BHT 0.01%) | 96.5 | 92.6 | 3.9 |
| PVA-PEG-Copolymer | 96.3 | 93.6 | 2.7 |

Tight primary packaging (e.g. aluminium laminate pouches) can be used to protect the wafers from humidity. In that case, the stability is generally improved. However, the stabilizing effect of the INA-PEG-Copolymer was confirmed in tight primary packaging even at high drug concentrations as reported in the table below.

Drug Content and Impurities of ERβ Selective Agonist in HPMC as a Matrix Polymer Containing BHT (0.01%) as Stabilizer Before and after 1 Month Storage in Tight Primary Packaging (Drug Conc. in the Polymer Matrix: 2.5%) Compared to a Unit Dosage form According to the Present Invention Comprising PVA-peg-copolymer.

| | after 1 month storage | | |
|---|---|---|---|
| START | 25° C. | 30° C. | 40° C. |

DRUG CONTENT, %

| | | | | |
|---|---|---|---|---|
| PVA-PEG-Copolymer | 96.3 | 96.3 | 96.3 | 96.1 |
| HPMC | 96.3 | 95.8 | 95.7 | 95.6 |

IMPURITIES, %

| | | | | |
|---|---|---|---|---|
| PVA-PEG-Copolymer | 3.7 | 3.7 | 3.7 | 3.9 |
| HPMC | 3.7 | 4.2 | 4.3 | 4.4 |

The drug delivery systems in the form of thin water-soluble films (wafers), comprising a PVA-PEG-copolymer offer superior stability of the active ingredient, particularly in the case said active ingredient being a steroid in which the positions 6 and 7 of the steroidal skeleton are both a —CH₁₂—. This allows longer shelf life and higher reliability of the dosage form.

### Acceptability of the Drug Delivery System (Wafer) According to the Invention

The acceptability of the wafer according to the invention was tested independently from the active ingredient. This approach allowed an evaluation of the dosage form on parameters which were therefore totally independent from those derived from the incorporation of the active ingredients allowing an overall selection of the preferred form. Pharmaceutical formulations directed to the oral cavity, and in particular wafers with improved mouthfeel are of paramount importance in a long term use and a high acceptability is required.

Wafers were prepared according to example 8f.

The acceptability of the placebo wafer formulations were evaluated in a human test panel (n=8) with respect to handling and administration. For both properties following features were evaluated:
Handling: —flexibility
Administration: —disintegration
   adherence (to palate)
   taste

Especially the mouthfeel, primarily represented by taste and disintegration time, seem to be a relevant parameter for the acceptability of the formulation for long term use.

The film thickness and flexibility were additionally quantified and correlated to the in-vivo evaluation.

The film thickness was measured by a MiniTest 600, Erichsen, Hemer, Germany The mechanical properties were quantified by measurement of the tensile strength and elongation (Zwick Material Testing, Ulm, Germany) and calculation of the modulus of elasticity, E, by following equation:

$$E = \frac{tensile strength}{elongation} = \frac{F / A}{Δ L / {L}_{0}}$$

with
E: modulus of elasticity (Young's modulus)
F: force (in N) applied to the object
A₀: original cross-sectional area through which the force is applied
ΔL: amount by which the length of the object changes
L₀: original length of the object

### Evaluation of Flexibility of Placebo Wafer Formulation

| | +++ | | |
|---|---|---|---|
| | (in all | ++ | + |
| Polymers/Additives | directions) | (90-180°) | (<90°) |
| HPMC | 6 | 2 | 0 |
| HPMC + 20% PG | 5 | 3 | 0 |
| HPMC + 20% TEC | 7 | 1 | 0 |
| HPMC + 5% gamma CD | 0 | 6 | 2 |
| HPMC + 2.3% | 6 | 2 | 0 |
| Propylgallat | | | |
| HPC (Klucel ® EF) | 8 | 0 | 0 |
| PVA-PEG Copolymer | 8 | 0 | 0 |

### Determination of the Film Thickness, the Mechanical Properties and Modulus of Elasticity of Placebo Wafer Formulation

| | Film | | | |
|---|---|---|---|---|
| | thickness, | | | E, |
| Polymers/Additives | μm | F, N | ΔL/L₀, % | MPa |
| HPMC | 68 ± 3 | 39.1 ± 3.0 | 10.8 ± 2.3 | 520 ± 71 |
| HPMC + 20% PG | 52 ± 4 | 25.8 ± 0.9 | 9.3 ± 0.3 | 447 ± 12 |
| HPMC + 20% TEC | 51 ± 3 | 15.5 ± 2.1 | 6.6 ± 0.9 | 385 ± 4 |
| HPMC + 5% gamma CD | 81 ± 10 | 37.6 ± 3.1 | 9.3 ± 0.7 | 414 ± 14 |
| HPMC + 2.3% | 51 ± 4 | 35.9 ± 1.7 | 10.6 ± 0.7 | 554 ± 35 |
| Propylgallat | | | | |
| HPC (Klucel ® EF) | 46 ± 2 | 6.3 ± 0.8 | 23.9 ± 8.1 | 51 ± 12 |
| PVA-PEG Copolymer | 49 ± 6 | 14.7 ± 1.2 | 25.8 ± 7.5 | 105 ± 43 |

Using HPC or PVA-PEG-Copolymer as a polymer matrix the resulting wafers were much more flexible than wafers containing HPMC as a polymer matrix, even those containing high amounts of plasticizers (e.g. propylene glycol (PG) or triethylcitrate (TEC) up to 20%). The measurement of the mechanical properties confirmed, that the modulus of elasticity was strongly decreased and the %-elongation (ΔL/L₀) much increased for HPC and PVA-PEG-Copolymer wafers compared to all other formulations.

Disintegration Time of Placebo Wafer Formulation after Administration (Normalized to a Wafer Thickness of 50 μm)

| Polymers/Additives | Time, s |
|---|---|
| HPMC | 18.7 |
| HPMC + PG | 14.2 |
| HPMC + TEC | 19.0 |
| HPMC + gamma CD | 17.7 |
| HPMC + Propylgallat | 19.1 |
| HPC (Klucel ® EF) | 28.8 |
| PVA-PEG Copolymer | 20.9 |

The mean value for the time until complete disintegration of the wafers was 20.3 seconds (S.D.: ±5.3 seconds). Additions of relevant amounts of liquid additives (e.g. plasticizers) resulted in a decrease of the disintegration time (e.g. HPMC vs. HMPC+PG). However, some polymers also prolonged the disintegration time remarkably (e.g. HPC). According to the results of the human taste panel rated, disintegration times of approx. 15-25 seconds, preferably about 20 seconds are perceived as pleasant.

Adherence to the Palate of Placebo Wafer Formulation after Administration

| Polymers/Additives | Very good | good | low |
|---|---|---|---|
| HPMC | 1 | 4 | 3 |
| HPMC + PG * | 3 | 4 | 0 |
| HPMC + TEC * | 2 | 5 | 0 |
| HPMC + gamma CD | 1 | 6 | 1 |
| HPMC + Propylgallat | 2 | 4 | 2 |
| HPC (Klucel ® EF) | 6 | 2 | 0 |
| PVA-PEG Copolymer | 5 | 3 | 0 |

* n = 7

Taste of Placebo Wafer Formulation after Administration

| | | tolerable | | in- |
|---|---|---|---|---|
| Polymers/Additives | enjoyable | (neutral) | bad | acceptable |
| HPMC | 1 | 5 | 2 | 0 |
| HPMC + PG | 0 | 6 | 2 | 0 |
| HPMC + TEC | 0 | 0 | 1 | 7 |
| HPMC + gamma CD | 0 | 2 | 6 | 0 |
| HPMC + Propylgallat | 0 | 4 | 4 | 0 |
| HPC (Klucel ® EF) | 3 | 6 | 0 | 0 |
| PVA-PEG Copolymer | 0 | 7 | 1 | 0 |

In general the taste of the formulation was related to the polymer matrix. Most additives altered the taste of the formulations significantly such that the taste turned bad, or even inacceptable (e.g. Triethylcitrate (TEC), gamma-Cyclodextrin (gamma CD)).

### Overview of Results of the In Vivo Evaluation of Placebo Wafer Formulations

| | Flexi- | Sticki- | Disintegra- | | |
|---|---|---|---|---|---|
| Polymers/Additives | bility | ness | tion time | Adherence | Taste |
| HPMC | + | + | + | − | + |
| HPMC + PG | + | − | − *1 | + | + |
| HPMC + TEC | + | + | + | + | −− |
| HPMC + gamma CD | − | + | + | + | − |
| HPMC + Propylgallat | + | + | + | + | + |
| HPC (Klucel ® EF) | ++ | − | − *2 | ++ | +++ |
| PVA-PEG Copolymer | ++ | − | + | ++ | ++ |

*1 too short,
*2 to long

The overall evaluation of the results revealed surprisingly a correlation of the perceived taste of the formulations to their flexibility, as seen for HPC and PVA-PEG-Copolymer. Thus, the flexibility of the film seems to have a crucial impact on the perceivable taste.

Moreover, also the adherence to the palate was improved with improved flexibility of the formulations.

In conclusion, more flexible films will result therefore in wafers with higher acceptability by the patients due to a higher comfort during administration related to an improved taste and adherence to the mucosa.

The present wafers demonstrate improved mouthfeel and taste, by defining favourable thickness and elasticity, able to confer improved patient acceptability.

Therefore unit dosage forms (wafers) with improved acceptability according to the invention comprise a thickness preferably in the range of about 45 μm to 80 μm and have a modulus of elasticity <200 MPas or particularly <150 MPas and/or a %-elongation >15%, or particularly >20%. Preferably the modulus of elasticity should be in the range of 20-200 MPas, particularly 40-150 MPas and/or the %-elongation should be in the range of 15-100%, particularly 20-50%.

The disintegration time for unit dosage forms (wafers) with improved acceptability as defined above and with a thickness normalized to about 50 μm is preferably between about 15 and 25 seconds.

As reported above the acceptability of unit dosage forms (wafers) according to the invention was tested independently from the active ingredients comprised therein to evaluate parameters which are independent from the incorporation of active ingredients.

However, the unit dosage forms (wafers) comprising an active ingredients and PVA-PEG-Copolymer according to the example 1 to 7 as described above were manufactured to meet also the requirements of thickness, disintegration time, elasticity, elongation and the other mechanical and organoleptical properties conferring an improved acceptability as defined above.

1. A unit dosage form comprising a thin water-soluble film matrix, wherein said film matrix comprises
a) a polyvinyl alcohol-polyethylene glycol graft copolymer (PVA-PEG graft co-polymer) as a water-soluble matrix polymer;
b) an active ingredient being a steroid in which the positions 6 and 7 of the steroidal skeleton are both a —CH₂— residue; and
said film matrix has a thickness of less than 300 μm.

2. The unit dosage form according to claim 1, wherein said active ingredient is a steroidal estrogen in which the positions 6 and 7 of the steroidal skeleton are both a —CH₂— residue.

3. The unit dosage form according to claim 1, wherein said active ingredient is a steroidal estrogen in which the positions 6 and 7 of the steroidal skeleton are both a —CH₂— residue and with an OH group, an ester or an ether group in position 3 of the steroidal skeleton.

4. The unit dosage form according to claim 1, wherein said active ingredient is selected from the group of ethinylestradiol, estradiol estrone, mestranol, estriol, estriol succinate, estrone sulfate, 17β-estradiol sulfate, 17α-estradiol sulfate, estradiol valerate including therapeutically acceptable derivates thereof.

5. The unit dosage form according to claim 2, wherein said steroidal estrogen in which the positions 6 and 7 of the steroidal skeleton are both a —CH₂— residue is a 8β- or 9α-substituted estra-1,3,5(10)-triene as ERβ selective agonist.

6. The unit dosage form according to claim 5, wherein said steroidal estrogen is selected from the group of:
9α-Vinyl-estra-1,3,5 (10)-triene-3,16α-diol,
17β-Fluoro-9α-Vinyl-estra-1,3,5 (10)-triene-3,16α-diol,
18a-Homo-9α-vinyl-estra-1,3,5 (10)-triene-3,16α-diol,
16α-Fluoro-8β-vinyl-estra-1,3,5 (10)-triene-3,17α-diol,
16α-Fluoro-8β-vinyl-estra-1,3,5 (10)-triene-3,17β-diol,
16β-Fluoro-8β-vinyl-estra-1,3,5(10)-triene-3,17β-diol,
8β-Vinyl-estra-1,3,5(10)-triene-3,17β-diol,
including therapeutically acceptable derivates thereof.

7. The unit dosage form according to claim 6, wherein said steroidal estrogen is 17β-Fluoro-9α-vinyl-estra-1,3,5(10)-triene-3,16α-diol including therapeutically acceptable derivates thereof.

8. The unit dosage form according to claim 1, wherein said active ingredient is a steroidal progestin in which the positions 6 and 7 of the steroidal skeleton are both a —CH₂— residue.

9. The unit dosage form according to claim 8, wherein said steroidal progestin is selected from the group of levonorgestrel, norgestrel, norethindrone (norethisterone), dienogest, norethindrone (norethisterone) acetate, ethynodiol diacetate, norethynodrel, allylestrenol, lynestrenol, norgestrienone, ethisterone, promegestone, desogestrel, 3-keto-desogestrel, norgestimate, gestodene.

10. The unit dosage form according to claims 2, wherein said film matrix comprises a further active agent being a progestin.

11. The unit dosage form according to claim 10, wherein said progestin is particularly a 16,17-carbolactone derivative in particular drospirenone.

12. The unit dosage form according to claim 10, wherein said progestin is selected from the group of levonorgestrel, norgestrel, norethindrone (norethisterone), dienogest, norethindrone (norethisterone) acetate, ethynodiol diacetate, dydrogesterone, medroxyprogesterone acetate, norethynodrel, allylestrenol, lynestrenol, quingestanol acetate, medrogestone, norgestrienone, dimethisterone, ethisterone, chlormadinone acetate, megestrol, promegestone, desogestrel, 3-keto-desogestrel, norgestimate, gestodene, tibolone, cyproterone acetate.

13. The unit dosage form according to, claim 1, wherein at least one active ingredient is complexed with a cyclodextrin or combined with a protective agent.

14. The unit dosage form according to claim 1, wherein at least an active ingredient is complexed with a cyclodextrin and at least an active ingredient is combined with a protective agent.

15. The unit dosage form according to claim 13, wherein said active ingredient combined with a protective agent is dispersed in the form of microparticles within the film matrix.

16. The unit dosage form according to claim 1, wherein the polyvinyl alcohol-polyethylene glycol graft copolymer is more than 50%, or 60%, or 70%, or 80%, or 90% by weight of said dosage form.

17. The unit dosage form according to claim 1, wherein said film matrix also comprises at least a further water-soluble matrix polymer selected from the group of a cellulosic material, a synthetic polymer, a gum, a protein, a starch, a glucan and mixtures thereof.

18. The unit dosage form according to claim 2, comprising 1-5000 μg of said steroidal estrogen, or derivative thereof.

19. The unit dosage form according to claim 1, wherein said film matrix has a thickness of less than 200 μm, or of less than 100 μm.

20. The unit dosage form according to claim 1, wherein said film matrix has a surface area of 2-10 cm², or of 3-7 cm², or of 4-6 cm².

21. The unit dosage form according to claim 1, having a weight in the range of from 5-200 mg, or in the range of from 10-100 mg, or in the range of from 10-50 mg.

22. The unit dosage form according to claim 1, having has a modulus of elasticity <200 MPas or <150 MPas or <100 MPas.

23. The unit dosage form according to claim 1, having a %-elongation >15%, or >20%.

24. The unit dosage form according to claim 1, wherein said dosage form comprises an absorption enhancer.

25. The unit dosage form according to claim 24, wherein said absorption enhancer is dissolved or dispersed in the film matrix.

26. A unit dosage form as defined in claim 1 for use as a medicament.

27. A unit dosage form comprising a thin water-soluble film matrix, wherein said film matrix comprises
a) a polyvinyl alcohol-polyethylene glycol graft copolymer (PVA-PEG graft co-polymer) as a water-soluble matrix polymer;
b) an active ingredient
said film matrix has a thickness of less than 100 μm.

28. The unit dosage form according to claim 27, having a modulus of elasticity in the range of 20-200 MPas.

29. The unit dosage form according to claim 27, having a %-elongation in the range of 15-100%.

30. The unit dosage form according to claim 27, wherein with a thickness normalized to about 50 μm the disintegration time of said unit dosage form is between about 15 and 25 seconds.

31. The unit dosage form according to claim 27, having a surface area of 2-10 cm², or of 3-7 cm², or of 4-6 cm².

32. The unit dosage form according to claim 27, having a weight in the range of from 10-50 mg.
